(19) United States
(12) Patent Application Publication
(10) Pub. No.: US 2009/0099568 A1
(43) Pub. Date: Apr. 16, 2009

(54) DEVICE AND METHOD FOR VARIABLY ADJUSTING INTERVERTEBRAL DISTRACTION AND LORDOSIS

(76) Inventors: David Lowry, Holland, MI (US); Desmond O'Farrell, Grand Rapids, MI (US); Scott Tuinstra, Holland, MI (US); Roger Veldman, Hudsonville, MI (US)

Correspondence Address: SHAY GLENN LLP, 2755 CAMPUS DRIVE, SUITE 210, SAN MATEO, CA, 94403, US

(21) Appl. No.: 12/188,131

(22) Filed: Aug. 7, 2008

Related U.S. Application Data
(60) Provisional application No. 60/954,507, filed on Aug. 7, 2007.

(51) Int. Cl. A61B 17/58; A61F 2/44

(52) U.S. Cl. 606/90; 623/17.11

(57) ABSTRACT

A removably insertable surgical apparatus is configured for insertion between adjacent vertebrae during spinal surgery and adjusted in-situ to produce varying degrees of distraction, neural-decompression, lordotic, kyphotic and/or scoliotic adjustment in the spine.

### CROSS REFERENCE TO RELATED APPLICATIONS

This application claims benefit to U.S. Provisional Application No. 60/954,507 filed on Aug. 7, 2007, entitled, “DEVICE AND METHOD FOR VARIABLY ADJUSTING INTERVERTEBRAL DISTRACTION AND LORDOSIS.”

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

This invention pertains to the apparatus and surgical method used to distract spinal vertebrae and to adjust the lordotic curvature of the spine. More particularly the invention relates to the design of a single intervertebral device which permits the in-situ adjustment of both distraction distance and lordotic angle to be achieved. While the cervical spine is the initial targeted use of the invention, variants thereof are anticipated for use in the thoracic and lumbar spinal regions.

### BACKGROUND OF THE INVENTION

Surgical procedures to relieve pain, decompress neural structures or to increase the stability of the spinal structure are common in the practice of spinal surgery.

Anterior or lateral approaches for spinal surgical procedures often employ the common practices of inducing axial separation of adjacent vertebral segment (vertebral distraction) for the purposes of neural de-compression and adjustment of the relative angle of the vertebral segments so as to establish and maintain a desired curvature within the spine, such as the lordotic (or forward bending) curvature of the cervical spine when viewed from a lateral (side) view.

The distraction process is one wherein the surgeon mechanically induces a separation of adjacent vertebrae in order to relieve neural compression and the associated pain and to prepare an interdiskal volume sufficient to receive an interbody implant of a substitute tissue material. Currently available distraction systems include the Caspar system supplied by Aesculap of San Francisco or the Cloward system by Cloward of Honolulu, Hi.

Care must be taken by the surgeon to avoid excessive distraction (over distraction) of the vertebrae as this can result in post surgical pain due to injury to the posteriorly located facet joints. Insufficient distraction (under distraction) can lead to the lack of a tight fit between any subsequently inserted intervertebral body implant and the vertebral bodies themselves.

Most of the currently available distraction techniques require a subjective assessment to be made by the surgical team as to the degree of distraction actually attained and as to when the point of over-distraction is being approached. Current distraction systems also fail to provide a quantifiable and controlled correction to a desired curvature in the spine, such as the previously mentioned lordotic curvature of the cervical spine.

In the instances where vertebral fusion or dynamic stabilization (such as with artificial disc replacement) is the desired clinical outcome, the distraction process further allows for improved surgical access to the vertebral end plate tissue, which must be removed or prepared prior to insertion of the prescribed interbody implant. In the instance of a fusion implant, end plate tissue is often removed in order to expose cancellous bone tissue to said implant, facilitating bone growth into or through said implant. In the instance where the implant is an artificial disc, end plate tissue is removed in order to assure intimate mechanical engagement of said implant device with the vertebral segments.

Artificial cervical discs include the Bryan and the Prestige systems (both by Medtronic of Minneapolis, Minn.) and the Prodisc-C system (by Synthes of West Chester, Pa.). Interbody implants for fusion include cadaveric allograft iliac strut or fibular implants (such as those supplied by the Musculoskeletal Foundation) or semi-synthetic implants such as the Bioplex system (by Biomet of Warsaw, Ind.) or metallic or polymeric implants such as those supplied by Biomet, Synthes, Medtronic and others.

When viewed from the side (or lateral) position, a normal, healthy spine has a natural varying forward bending curvature referred to as lordosis in the cervical and lumbar regions and a backward bending curvature referred to as kyphosis in the thoracic region. The actual amount of lordosis varies by location within the spine and further varies from patient to patient. Lordosis or kyphosis is typically described as the included angle between adjacent vertebrae, and this angle can vary from 3 degrees to 20 degrees in a healthy spine, depending upon the location within the spinal column and the specific physiology of the patient. Degeneration of the vertebral tissue or of the intervertebral disk tissue can either result from the loss of natural lordosis or be the cause of it, further compromising patient comfort and patient function.

Another spinal condition amenable to treatment by this invention is scoliosis, in which abnormalities of spinal curvature can be present when viewing the spine from the frontal (antero-posterior) perspective or from the side (lateral) perspective. Abnormal rotations within the axis of the spine may also occur with scoliosis.

Examples of optimal surgical outcomes include those wherein neural decompression is achieved without over-distraction of the vertebrae, intervertebral spacing is restored, lordotic curvature is restored, scoliotic curvature is restored and intervertebral fastening of the adjacent vertebrae occurs successfully. A successful outcome is further aided by minimizing the number of entries into, and exits from, the surgical incision, thereby minimizing the instances, and risk, of tissue damage.

As disclosed in the published art, various attempts to achieve this end effect have been made. These attempts typically involve the forcible insertion of wedge type instruments, devices or inserts into the intervertebral space. Distraction and lordotic adjustment occur to varying degrees as a result of the included angle of the inserted wedge, the cranio-caudal size of said wedge at the point of impingement on the adjacent vertebral surfaces at the inserted depth and as a result of the general physical condition of the patient. In most instances these procedures require a trial and error approach by the surgical team, and typically require inserting and removing multiple distractor wedges of varying included angles and varying sizes until an acceptable outcome is finally achieved.

The disclosed or published art in regard to distraction apparatuses and methods can generally be categorized into three distinct groups: mechanisms mounted onto or into adjacent vertebrae which are adjusted by the surgeon to attain distraction, wedge type instruments or intervertebral inserts and adjustable interbody fusion devices.

US Patent Application No. 2004/0106927 A1 to Ruffer et al. describes a distraction device having a scissors like action to induce distraction by insertion of one end of the device into the intervertebral space and engaging on the end plate surfaces of the adjacent vertebrae and then compressing the protruding handles of the device so as to induce axial separation of the vertebrae involved

U.S. Pat. No. 5,059,194 to Michelson discloses a four leg distractor device wherein the legs are inserted into the intervertebral space with two legs impinging upon each of the vertebrae. The space between opposing sets of legs is mechanically adjusted resulting in distraction of the vertebrae.

These, and other similar distraction devices and apparatus, produce distraction that is essentially axial in nature and do not facilitate substantial, controlled lordotic adjustment.

US Patent Application No. 2006/0036247 A1 to Michelson discloses the use of a removable distractor insert which is generally conical in nature at the insertion point of the device and generally cylindrical thereafter. A further generally wedge shaped short distractor is also disclosed. The distractor devices disclosed are of fixed included angles and fixed dimensions and require the use of additional specialized tools to insert the distractor device so as to achieve distraction and to subsequently remove the distractor insert.

U.S. Pat. No. 6,224,599 to Baynham et al. discloses a wedge shaped insertion and distraction tool used to insert a removable access port between the adjacent vertebrae to facilitate the performance of surgical procedures. This wedge device described is of fixed included angle and is intended as an insertion tool for the purposes of inserting a removable access port between vertebral segments within the spinal column.

US Patent Application No. 2004/0215203 A1 and U.S. Pat. Nos. 6,270,498, 6,080,155, 6,770,074, 6,096,038, 5,797,909 and 5,5057,732 all to Michelson disclose and discuss the use of fixed included angle wedge type distractors for the purposes of distraction and lordotic adjustment.

U.S. Pat. No. 7,153,304 to Robie et al. discloses the use of a distractor insert with a tapered section for the purposes of restoring natural lordosis of the spine. This device is a fixed included angle wedge type device.

These, and similar wedge style devices have fixed included angles and rely upon the surface to surface contact between the vertebrae and the tapered surfaces of the device to induce distraction and lordotic adjustment. They are generally forcibly inserted between the vertebrae. Only a small amount of the insertion force actually produces distraction. The majority of the insertion force is normal to the axis of the spine. Only the component resulting from the wedge angle actually induces distraction, with the balance being counterproductive and potentially damaging to tissue.

U.S. Pat. No. 5,893,890 to Pisharodi discloses a permanently implanted interbody fusion device that is inserted between adjacent vertebrae, positioned and subsequently expanded by means of a screw mechanism, locking it in position between said vertebrae.

U.S. Pat. No. 5,984,922 discloses a permanently implanted interbody insert, placed between adjacent vertebrae and axially expanded by a screw mechanism in order to provide a permanent mounting means to which a posterior spinal fixation device is attached.

U.S. Pat. No. 6,102,950 discloses a wedge shaped intervertebral fusion device which is a permanently implanted intervertebral fixation device which is screwably expanded to assure permanent impingement and penetration of surface protrusions on said device into the vertebral bone tissue in order to restore lordosis.

U.S. Pat. Nos. 6,648,917 B2 and 6,562,074 both to Gerbec et al. disclose bone fusion implants which are sizably adjusted after being permanently inserted between adjacent vertebrae.

Devices of this type are intended as permanent interbody implants and are not intended for use as temporary distraction devices which will be removed prior to the completion of the surgical procedure. These devices demonstrate the desirability of having available devices which can be adjusted in-situ so as to accommodate the variations encountered during orthopedic and neurological surgical procedures.

According to one aspect of the invention, a single distractor device can be easily inserted without substantial restriction or impediment into an intervertebral space to a prescribed depth therein, can be adjusted in-situ by the surgeon to induce both axial vertebral distraction and also lordotic, kyphotic, or scoliotic correction in a controlled and measurable manner without the need for forceable impact onto, or impingement into the vertebral tissue and can be subsequently removed from the vertebral space

According to other aspects of the invention, a distraction device may be provided which is faster, safer and more efficacious than those currently disclosed or available.

### SUMMARY OF THE INVENTION

According to aspects of the present invention, an intervertebral distractor device may be provided comprising an assembly of three primary components: a body; a screw mechanism; and a sliding expansion insert.

In one exemplary embodiment the body of the device has two substantially parallel external walls disposed to fit into the space between adjacent vertebrae and to be in loose, general contact with the vertebral endplates after insertion. The two parallel walls may be rigidly connected by a cross member near the anterior end of the device, the cross member being substantially perpendicular to the parallel walls. The cross member may have protrusions beyond the parallel walls of sufficient dimension so as to restrict the maximum depth of device insertion into the spinal column by engaging with the anterior surfaces of the adjacent vertebrae.

In this embodiment, each of the parallel walls has within it a hingeably attached flap, the flap being attached to the wall, proximal to the posterior or distal edge of the wall. In the same embodiment, this hinge may be a living hinge integrally manufactured from the same material as the wall, it may be a secondarily assembled hinge or it may be provided by injection molding, insert injection molding or co-injection molding processes.

The interconnecting cross member may receive through it a threaded cylinder located generally central to said cross member. The centerline of the threaded cylinder is parallel with the side walls of the device. In this embodiment, the threaded shaft is screwably inserted through the cross member and engages the expansion insert, which is located between the expansion flaps. This expansion insert has a width that is nominally greater than the dimension between the internal surfaces of the flaps in the side walls.

Subsequent to assembly, the screw device may be rotated in the threaded body cylinder causing the expansion insert to move axially in a proximal to distal direction (in an anterior to posterior direction relative to the spine) resulting in a sliding movement and interferential engagement between the flaps and the expansion insert. This interference causes the flaps which are engaged on the vertebral end plates to rotate or flex in an outward direction, relative to the hinge axes resulting in an increase in the distance between the vertebrae and in a change in included angle of the vertebrae. This movement is substantially transferred to the adjacent vertebrae which results in both distraction and lordotic adjustment to the vertebrae.

In another embodiment of the invention the external profile of the expansion insert is contoured, resulting in variable rates of distraction and lordotic adjustment as it translates along the contact surfaces of the hinged flaps.

In other embodiments the rigid, non-moving portions of the side wall are interconnected at the posterior, distal end to increase the rigidity of the device and prevent converging flexure of the side wall during the adjustment of the expansion insert.

In any embodiment the device may be manufactured from a metal such as titanium, stainless steel or other medical instrument grade metal, the device may be manufactured from a medical grade polymer such as PEEK, it may be a combination of metals and polymers, or made from other suitable materials.

In some embodiments of the device the expansion flap and integral hinge may be located proximal to the anterior vertebral surface and the expansion flap slot may be located in the posterior region. In these embodiments movement of the adjustment means results in a posterior to anterior movement of the expansion insert.

In any embodiment of the device the adjustment means may be coupled with an external control and measurement device which provides precise feedback to the surgeon, this feedback being a measurement of distraction distance, distraction angle, force applied or any combination thereof.

In some embodiments of the present invention, the method of insertion for the correction to a desired lordotic or kyphotic curvature is from the anterior aspect of the spinal column. Just as this invention would permit the correction of a lordotic or kyphotic curvature abnormality when implanted from an anterior approach, its implantation or placement from a lateral approach would correct scoliotic abnormalities of spinal curvature when viewed from a frontal or antero-posterior perspective. A method of inserting the device into the disc space from an oblique angle anywhere between an anterior approach and a lateral approach would facilitate the simultaneous correction of curvature abnormalities in both the frontal and side perspectives.

In some embodiments of the present invention, a vertebral distraction device is provided comprising a first element configured to contact a first endplate of a vertebral body, a second element spaced apart from the first element and configured to contact an opposing second vertebral endplate located on an adjacent vertebral body, and an adjustment mechanism configured to alter the spacing between the first and second elements. The device may contact each of the endplates on at least three non-collinear points so as to control an included angle of the endplates.

In some of the above embodiments, the vertebral distraction device may comprise an adjustment mechanism configured to simultaneously increase the spacing and an included angle between the first and second endplates. The adjustment mechanism may comprise a lead screw, which may have an axis that generally bisects the included angle of the vertebral endplates when in use. In some embodiments, at least one of the first and second elements comprises a flap hingedly connected to a body of the device. The at least one flap may be connected to the device body near a distal end of the device body, or near a proximal end of the device body. In some embodiments, each of the first and second elements comprises a flap hingedly connected to a body of the device.

In some embodiments, a vertebral distraction device may be provided with an adjustment mechanism comprising an expansion insert located between the first and second elements described above. The adjustment mechanism may be configured to move the expansion insert laterally with respect to the first and second elements. The adjustment mechanism may further comprise a screw that causes the expansion insert to move when the screw is turned. The expansion insert may be configured to move at least one of the first and second elements outwardly as the expansion insert moves laterally. The expansion insert may be configured to move at least one of the first and second elements outwardly at a non-uniform rate as the expansion insert moves laterally. In some embodiments, a vertebral distraction device may be provided with an insertion stop configured to abut against an outer surface of at least one adjacent vertebral body to prevent the device from penetrating an intervertebral space beyond a predetermined depth.

According to aspects of the present invention, a surgical kit may be provided which comprises at least one vertebral distraction device as described above, and a spinal plate configured for implanting across two or more adjacent vertebral bodies. The plate may comprise at least one feature configured to align the plate relative to a mating feature of the at least one vertebral distraction device. In some embodiments, the aligning feature on the plate is an aperture and the mating feature of the at least one vertebral distraction device is a portion of a body of the device. The aperture may be sized to permit removal of the device through the aperture. Any of the above surgical kits may further comprise two or more of the vertebral distraction devices.

In some embodiments of the present invention, a non-implantable vertebral distraction device may be provided which comprises a first wall and, a second wall spaced apart from and generally parallel to the first wall. The device may further comprise a cross member interconnecting an end of each of the first and second walls, with the cross member being oriented generally perpendicular to the first and second walls. The device may further comprise a first flap hingedly connected to the first wall and pivotable between a first position and a second position laterally outward from the first position, and a second flap hingedly connected to the second wall and pivotable between a third position and a fourth position laterally outward from the third position. An expansion insert may be located between the first and second walls and movable in a direction generally parallel to the first and second walls between a fifth position and sixth position. The device may further comprise an adjustment mechanism coupled to the cross member for driving the expansion insert between the fifth and sixth positions, wherein the movement of the expansion insert from the fifth position to the sixth position causes the first flap to move from the first position to the second position and causes the second flap to move from the third position to the fourth position.

In some of the embodiments described above, the fifth position is located between the sixth position and the cross member. In other embodiments, the sixth position is located between the fifth position and the cross member. The cross member may further comprise at least one protrusion extending in a direction generally perpendicular to the first and second walls and configured to abut against an outer surface of a vertebral body to limit a penetration depth of the device into an intervertebral space. In some embodiments, the adjustment mechanism comprises a lead screw, a plunger and/or a ratchet. In some embodiments, the device further comprises a distal member spaced apart from and generally parallel to the cross member. This distal member interconnects an end of each of the first and second walls opposite the cross member. An end member may be arranged generally perpendicular to each of the first wall, the second wall, and the cross member. This end member may interconnect a side portion of each of the first and second walls.

According to aspects of the present invention, methods of changing the respective orientation of two adjacent vertebrae may be used, wherein the vertebrae each have an opposing endplate defining an intervertebral space between the vertebrae. In some embodiments, the method comprises the steps of engaging each of the two vertebrae with a distraction device such that a relative axial distance between the two endplates and an included angle between the two endplates are controlled. The methods may further comprise the step of varying both the relative axial distance and the included angle between the two endplates by moving two portions of the distraction device relative to one another.

In some of the above methods, the engaging step comprises inserting the distraction device at least partially into the intervertebral space, and each of the two portions of the device contacts one of the vertebral endplates on at least three non-collinear points. At least one of the two portions of the device may comprise a flap hingedly connected to a body of the device. The device may comprise an adjustment mechanism having a movable expansion insert located between the two portions of the device. In some embodiments, the engaging step comprises inserting the distraction device at least partially into the intervertebral space until a stop on the device abuts against an outer surface of at least one of the vertebrae and prevents further insertion of the device. The above methods may further comprise aligning a vertebral plate with the distraction device and attaching the plate to at least one of the vertebrae after the engaging and varying steps. The varying step may be used to adjust one of a lordotic, kyphotic or scoliotic angle of a portion of a spine. The methods may be used to adjust these angles individually or simultaneously.

### BRIEF DESCRIPTION OF THE DRAWINGS

FIG. 1 is an exploded perspective view showing the three primary components of an exemplary embodiment of the invention;

FIG. 2A is a perspective view showing the device of FIG. 1 in its assembled condition;

FIGS. 2B, 2C and 2D are graphical illustrations of an alternative embodiment of receiving means;

FIG. 2E is a side cross-section view of a portion of an extraction device.

FIG. 3 is a cross section view taken through Plane A-A of FIG. 2 showing the device components in their assembled, pre-operative positions;

FIG. 4 is a cross section view similar to FIG. 3 showing the device components in their adjusted position with the expansion flaps expanded;

FIG. 5 is a perspective view of the body showing an alternative embodiment having the vertical side walls connected at the posterior, distal end;

FIG. 6 is a perspective view from the direction of View B in FIG. 2 of the body showing an alternative embodiment having the vertical side walls connected at their edges by a vertical wall;

FIG. 7 is a perspective view of the body showing an alternate configuration of the expansion flaps, having protrusions on the external surfaces of the flaps for engaging compressively on the vertebrae upon insertion;

FIG. 8 is a cross section view of FIG. 7 taken through Plane C-C showing protrusions on the expansion flap external surfaces;

FIG. 9 is a cross section view similar to FIG. 8 showing protrusions on the expansion flap external surfaces in a compressed position after insertion between vertebrae;

FIG. 10 and FIG. 11A are a graphic illustration of an exemplary insertion and adjustment procedure, with FIG. 11A demonstrating the introduction of lordotic angle ALPHA and distraction equivalent to distance d2 minus d1;

FIG. 11B is a perspective view showing the exemplary distraction device in an expanded state;

FIG. 12A-12C graphically illustrate the use of a lobed expansion insert to create continuously variable or non-linear adjustment;

FIG. 13 is a graphical illustration of the varying lordotic adjustment and distraction that can be achieved as a result of linear travel of the expansion insert.

### DETAILED DESCRIPTION OF THE INVENTION

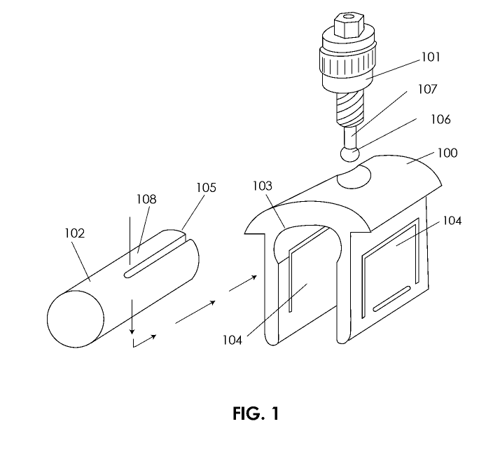

FIG. 1 is an exploded view of the assembly constructed according to aspects of the invention. The assembly includes a body 100, an adjusting screw 101, and an expansion insert 102. The body 100 is adapted to receive and retain the expansion insert 102 in a measured receiving chamber 103 which positions the wedge 102 relative to a pair of opposing expansion flaps 104. The expansion wedge 102 is slideably engaged on the screw device 101 by means of a receiving slot 105 through which the non-threaded end 107 of the screw 101 slides until it engages the controlled stop surface 108, assuring its correct location relative to the expansion flaps.

In an alternate embodiment of this device, not shown, the expansion insert 102 is omitted from the assembly and the distal end 106 of the adjustment mechanism 101 slideably engages directly with the expansion flaps. This embodiment may be used in certain situations where the intervertebral distance is small, such as that encountered in the upper cervical region or in situations where there has been substantial degeneration of the intervertebral disk.

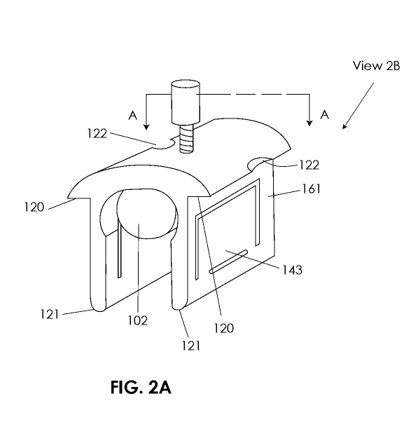

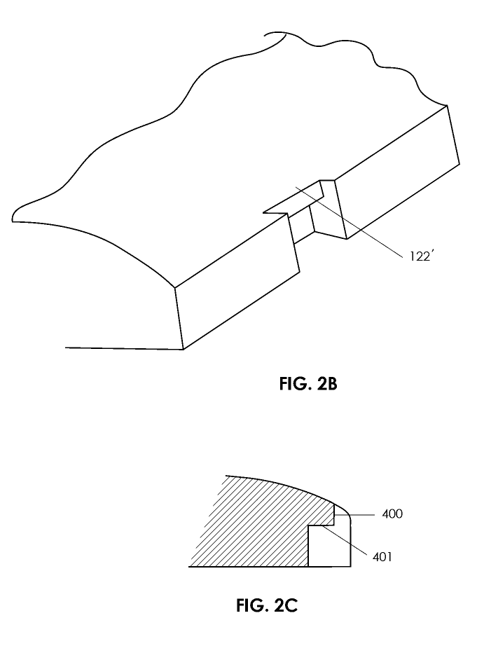

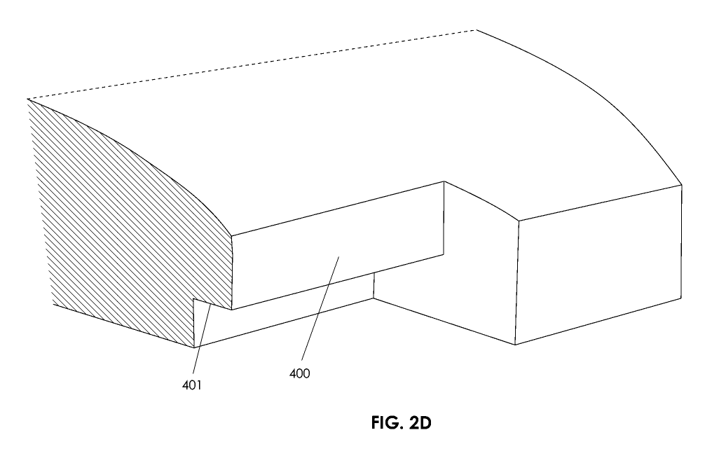

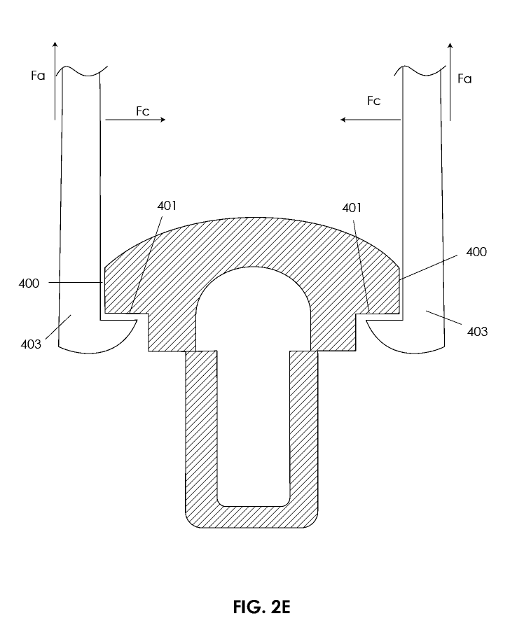

FIG. 2A is a perspective view of the components in their assembled condition and shows the referenced protrusion 120. The distance between point 121 and surface 120 defines the maximum insertion depth of the device so as to guarantee that the device cannot be inserted beyond a safe depth into the spinal column. FIG. 2 further shows receiving means 122 for accepting an extraction tool to facilitate removal of the device.

FIGS. 2B, 2C and 2D are graphical illustrations of an alternative embodiment of receiving means, the receiving means 122′ comprising one or more recessed volumes within the body of the device disposed to receive an extractor device which compressively engages surface 400. The device is thereafter extracted by application of an axial force on surface 401.

FIG. 2E is an illustration of an extraction device having arms 403 compressively engaged with the distractor device by the application of a compressive force Fc. The device is extracted from the intervertebral volume by the application of an axial force Fa.

In one embodiment of an extraction device, a scissors action is used whereby the compressive force Fc and the axial force Fa can be simultaneously applied to the device.

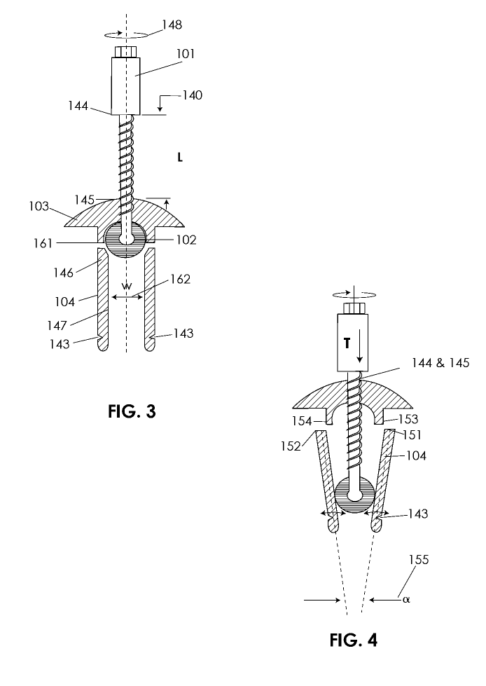

FIG. 3 shows a cross section of the device in FIG. 2, taken through plane A-A in FIG. 2. The exemplary device is in its assembled, pre-operative condition with the expansion insert 102 in the unadjusted or retracted position and the expansion flaps 104 in their natural unexpanded position. The adjusting screw 101 is shown with a mechanical stop 144 intended to engage on the surface of the body 145 in order to prevent over distraction of the vertebrae by limiting the axial travel of the expansion insert 102. The external diameter 141 of the expansion insert 182 is larger than the distance between the internal surfaces 147 of the adjusting flaps in their natural unexpanded state.

Rotational adjustment 148 of the screw mechanism 101 results in downward travel of the expansion insert 102 such that initial engagement occurs between its external diameter 141 and points 146 on the expansion flaps. This engagement provides tactile feedback that the point of initial expansion has been reached and any further adjustment of the screw mechanism will induce expansion of the device flaps 104 between the adjacent vertebrae. In the embodiment shown, rotational adjustment 148 of the screw mechanism 101 may be accomplished by hand or with the use of a tool.

FIG. 4 shows a cross section of the device, similar to FIG. 3. In FIG. 4, the device is in its assembled and expanded condition with the expansion insert 102 at the maximum allowable position, defined by the contact of points 144 and 145, and the expansion flaps 104 are in their expanded position having been rotated relative to their hinge points 143. Distraction is achieved as the dimension between the expanded flaps 104 described as the distance between points 151 and 152 is greater than the initial body dimension described as the distance between points 153 and 154. Further, lordotic correction has been introduced by the rotational adjustment of the flaps 104 with respect to their hinge points 143 introducing a corrective angle ALPHA.

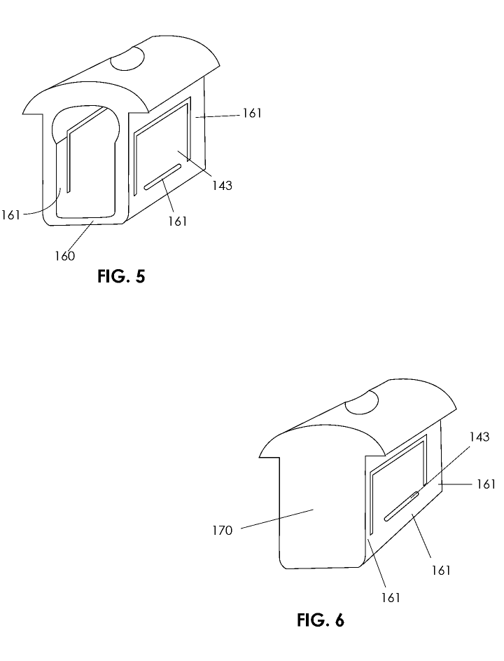

FIG. 5 is a perspective view showing an alternate embodiment of the device body 100 illustrated in FIG. 1 wherein the parallel walls are interconnected at their posterior (distal) ends by a wall 160 for the purposes of increasing the rigidity of the non-expanding wall portions 161 and allowing for greater adjustment of the vertebrae without the risk of converging flexure of the unrestrained wall 161 which may occur in the device shown in FIG. 1.

FIG. 6 is a perspective view from direction of VIEW B in FIG. 2 showing another alternate embodiment of the device body 100 illustrated in FIG. 1 wherein the parallel walls are interconnected by a vertical wall 170 for the purposes of increasing the rigidity of the non-expanding wall portions 161 and allowing for greater adjustment of the vertebrae without the risk of converging flexure of the unrestrained wall 161 which may occur in the device shown in FIG. 1.

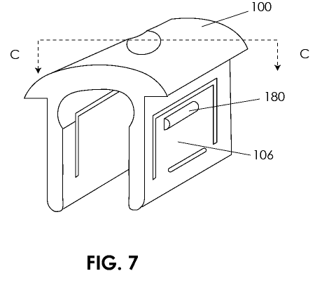

FIG. 7 is a perspective view of the device body 100 with the expansion flaps 104 having one or more protrusions 180 on the vertebral engaging walls designed to flex the expansion flaps 104 inwardly and introduce compressive loading on said expansion flaps 104 upon insertion of the device between adjacent vertebrae. This compressive loading is intended to assure an adequate fit of the device in the intervertebral space and to retain the device in its inserted position during initial engagement of the expansion insert 102. The compression of the extension flaps further increases the effective distraction distance and lordotic corrective angle that can be achieved.

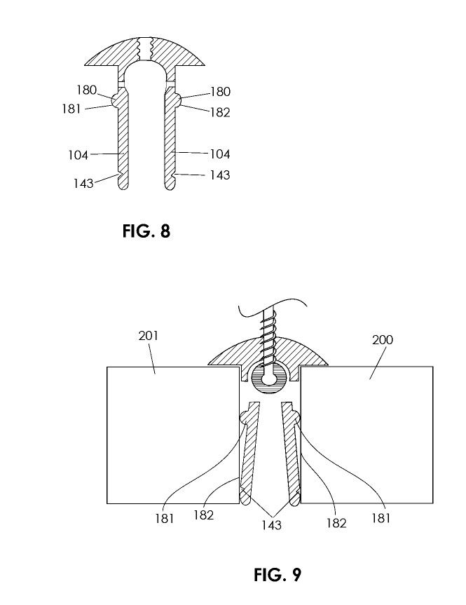

FIG. 8 shows a cross section view of the device in FIG. 7 taken through plane C-C and demonstrates the compression protrusions 180 on the exterior surface of the expansion flaps 104.

FIG. 9 is a cross section view of the device shown in FIG. 8 after insertion between adjacent vertebrae 200 and 201. Engagement of the compression protrusions 181 with the vertebral end plates 182 results in a compression load being applied to the flaps 104 causing their inward flexure the hinge points 143. As a consequence the device becomes centered in the intervertebral space and the resulting interference between the protrusions 181 and the disk end plates 182 acts to locate and retain the device during initial adjustment.

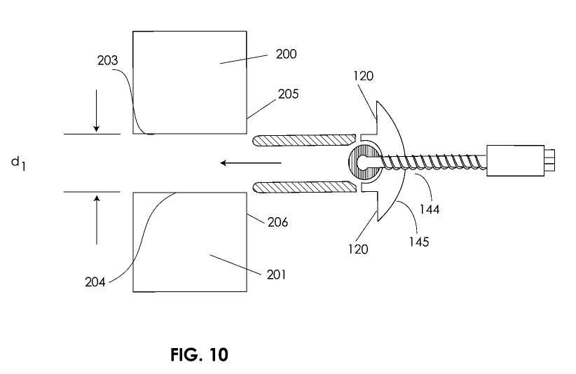

Referring to FIG. 10, the assembled device is inserted between endplates 203 and 204 of adjacent vertebrae 200 and 201 in the unexpanded state. To facilitate insertion, a partial diskectomy may be performed. The device is of a width equal to or slightly less than the interdisk space, allowing for easy insertion to the prescribed depth. Optimal insertion has been achieved when posterior surfaces 120 of the cross member protrusions come in contact with the anterior surfaces 205 and 206 of the vertebrae 200 and 201.

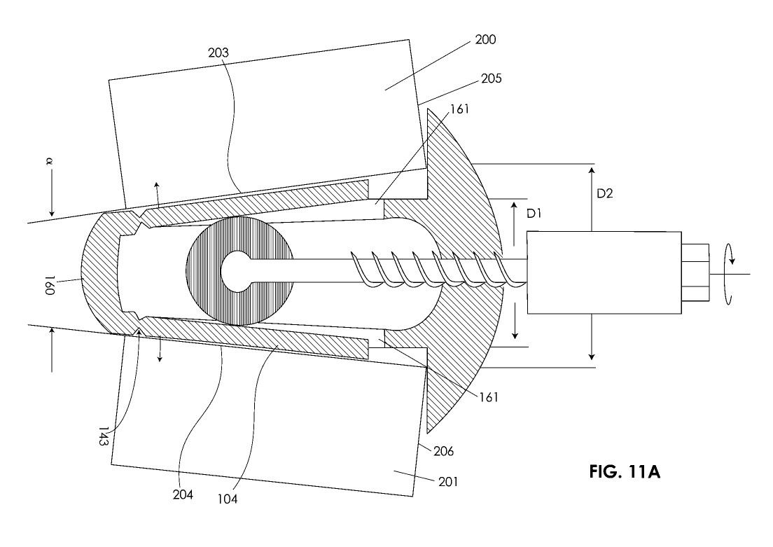

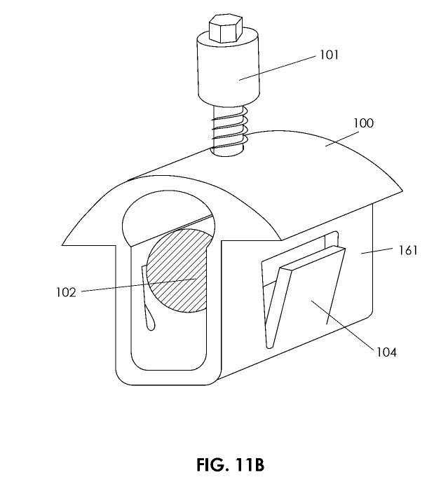

Referring to FIG. 11A the screw mechanism 101 is rotationally adjusted causing the expansion insert 102 to travel in a posterior direction along the expansion flaps 104. This travel induces angular or rotational change of the flaps 104 relative to the hinge point 143 producing a change in the intervertebral distance (distraction) and in the relative angles of the adjacent vertebrae described as angle “α”. The amount of distraction and lordotic adjustment produced is proportional to the travel of the expansion insert 102 along the interior surfaces of the expansion flaps 104 and is threadably adjusted by the surgeon while the device remains in-situ. The maximum attainable travel is restricted by the impingement of shoulder 144 on the screw mechanism 101 onto the anterior surface 145 of the body 100.

FIG. 11B shows another view of the exemplary distraction device in an expanded state.

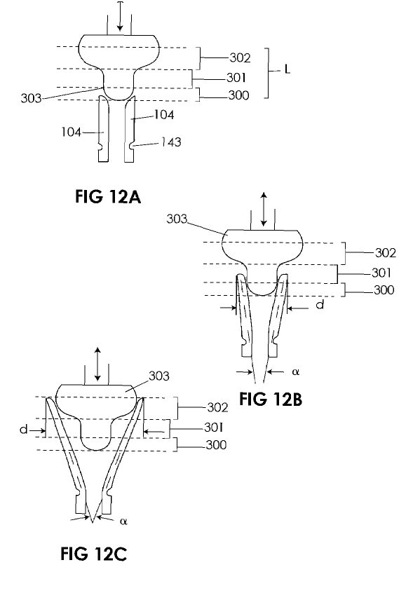

Referring to FIGS. 12A, 12B and 12C, the external profile of an expansion insert can be varied to produce varying and/or non-linear distraction rates and/or varying and non-linear angle change rates of lordotic adjustment. Distraction and lordotic adjustment result from the tangential engagement of the outer contour of said insert on expansion flaps 104. By varying the outer profile of the expansion insert 303 the effective expansion width of the insert 303 can be adjusted so that the initial distraction rates are relatively fast as indicated by the engagement over the perimeter length 300 of the adjustment insert 303 so as to assist the surgeon in attaining initial distraction. Fine adjustment can again be attained over the length 301 and rapid angular adjustment can be produced over the length 302.

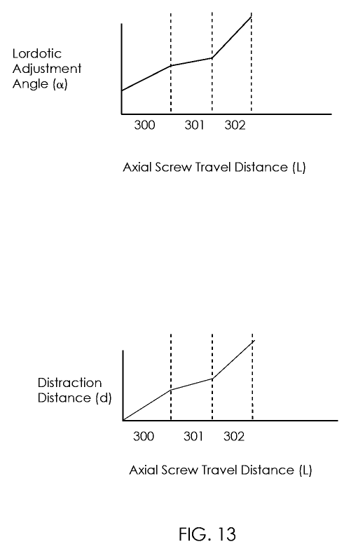

FIG. 13 is a graphical illustration of the relative distraction distance and lordotic angular adjustment range that can be produced using the exemplary tri-lobular expansion insert 303 adjusted over the lengths 300, 301 and 302 respectively.

Counter rotation of the screw mechanism results in the release of the distracting and engaging forces between the device and the vertebrae to facilitate easy removal of the device after the desired distraction and lordotic adjustment has been secured in place such as by an external template, frame, plate or other devices. Said external device may obtain a relative location from the body cross member prior to attachment to the adjacent vertebrae.

Such a frame device is disclosed in co-pending U.S. patent application Ser. No. 11/855,124 entitled “Implantable bone plate system and related method for spinal repair” filed on Sep. 13, 2007, and associated provisional application No. 60/954,511 filed on Aug. 7, 2007.

Once the device is removed the surgeon has clear and un-impeded access to the distracted intervertebral space in order to perform the necessary surgical procedures.

While certain forms and embodiments of the invention are illustrated herein, it is understood that the invention is not limited to the disclosed forms or arrangements described and shown. It will be apparent to those skilled in the art that various changes may be made without departing from the scope of the invention and the invention is not limited to what is shown and described in the specifications and drawings herein. Specifically, it is anticipated that the inventions will apply to kyphotic or lordotic angle corrections throughout the spinal column. The inventions will also enable correction of a scoliotic curvature when viewed from an antero-posterior perspective, just as the invention enables a lordotic or kyphotic curvature correction when viewed from the side or lateral perspective. Additionally, a plunger, ratchet, or other type of adjustment mechanism may be substituted for the lead screw mechanism disclosed herein and still fall within the scope of the appended claims.

What is claimed is:

1. A vertebral distraction device comprising:
a first element configured to contact a first endplate of a vertebral body;
a second element spaced apart from the first element and configured to contact an opposing second vertebral endplate located on an adjacent vertebral body; and
an adjustment mechanism configured to alter the spacing between the first and second elements, wherein the device contacts each of the endplates on at least three non-collinear points so as to control an included angle of the endplates.

2. The vertebral distraction device of claim 1 wherein the adjustment mechanism is configured to simultaneously increase the spacing and an included angle between the first and second endplates.

3. The vertebral distraction device of claim 1 wherein the adjustment mechanism comprises a lead screw.

4. The vertebral distraction device of claim 3 wherein the lead screw has an axis that generally bisects the included angle of the vertebral endplates when in use.

5. The vertebral distraction device of claim 1 wherein at least one of the first and second elements comprises a flap hingedly connected to a body of the device.

6. The vertebral distraction device of claim 5 wherein the at least one flap is connected to the device body near a distal end of the device body.

7. The vertebral distraction device of claim 5 wherein the at least one flap is connected to the device body near a proximal end of the device body.

8. The vertebral distraction device of claim 1 wherein each of the first and second elements comprises a flap hingedly connected to a body of the device.

9. The vertebral distraction device of claim 1 wherein the adjustment mechanism comprises an expansion insert located between the first and second elements.

10. The vertebral distraction device of claim 9 wherein the adjustment mechanism is configured to move the expansion insert laterally with respect to the first and second elements.

11. The vertebral distraction device of claim 10 wherein the adjustment mechanism further comprises a screw that causes the expansion insert to move when the screw is turned.

12. The vertebral distraction device of claim 10 wherein the expansion insert is configured to move at least one of the first and second elements outwardly as the expansion insert moves laterally.

13. The vertebral distraction device of claim 10 wherein the expansion insert is configured to move at least one of the first and second elements outwardly at a non-uniform rate as the expansion insert moves laterally.

14. The vertebral distraction device of claim 1 further comprising an insertion stop configured to abut against an outer surface of at least one of the adjacent vertebral bodies to prevent the device from penetrating an intervertebral space beyond a predetermined depth.

15. A surgical kit comprising:
at least one vertebral distraction device according to claim 1; and
a spinal plate configured for implanting across two or more adjacent vertebral bodies, the plate comprising at least one feature configured to align the plate relative to a mating feature of the at least one vertebral distraction device.

16. The surgical kit of claim 15 further comprising two or more vertebral distraction devices according to claim 1.

17. The surgical kit of claim 15 wherein the aligning feature on the plate is an aperture and the mating feature of the at least one vertebral distraction device is a portion of a body of the device, and wherein the aperture is sized to permit removal of the device through the aperture.

18. A non-implantable vertebral distraction device comprising:
a first wall;
a second wall spaced apart from and generally parallel to the first wall;
a cross member interconnecting an end of each of the first and second walls, the cross member being oriented generally perpendicular to the first and second walls;
a first flap hingedly connected to the first wall and pivotable between a first position and a second position laterally outward from the first position;
a second flap hingedly connected to the second wall and pivotable between a third position and a fourth position laterally outward from the third position;
an expansion insert located between the first and second walls and movable in a direction generally parallel to the first and second walls between a fifth position and sixth position; and
an adjustment mechanism coupled to the cross member for driving the expansion insert between the fifth and sixth positions, wherein the movement of the expansion insert from the fifth position to the sixth position causes the first flap to move from the first position to the second position and causes the second flap to move from the third position to the fourth position.

19. The vertebral distraction device of claim 18 wherein the fifth position is located between the sixth position and the cross member.

20. The vertebral distraction device of claim 18 wherein the sixth position is located between the fifth position and the cross member.

21. The vertebral distraction device of claim 18 wherein the cross member comprises at least one protrusion extending in a direction generally perpendicular to the first and second walls and configured to abut against an outer surface of a vertebral body to limit a penetration depth of the device into an intervertebral space.

22. The vertebral distraction device of claim 18 wherein the adjustment mechanism comprises a lead screw.

23. The vertebral distraction device of claim 18 wherein the adjustment mechanism comprises a plunger.

24. The vertebral distraction device of claim 18 wherein the adjustment mechanism comprises a ratchet.

25. The vertebral distraction device of claim 18 further comprising a distal member spaced apart from and generally parallel to the cross member, the distal member interconnecting an end of each of the first and second walls opposite the cross member.

26. The vertebral distraction device of claim 18 further comprising at least one end member arranged generally perpendicular to each of the first wall, the second wall, and the cross member, the end member interconnecting a side portion of each of the first and second walls.

27. A method of changing the respective orientation of two adjacent vertebrae, the vertebrae each having an opposing endplate defining an intervertebral space between the vertebrae, the method comprising:
engaging each of the two vertebrae with a distraction device such that a relative axial distance between the two endplates and an included angle between the two endplates are controlled; and
varying both the relative axial distance and the included angle between the two endplates by moving two portions of the distraction device relative to one another.

28. The method of claim 27 wherein the engaging step comprises inserting the distraction device at least partially into the intervertebral space, and wherein each of the two portions of the device contacts one of the vertebral endplates on at least three non-collinear points.

29. The method of claim 27 wherein at least one of the two portions of the device comprises a flap hingedly connected to a body of the device.

30. The method of claim 29 wherein the device comprises an adjustment mechanism having a movable expansion insert located between the two portions of the device.

31. The method of claim 28 wherein the engaging step comprises inserting the distraction device at least partially into the intervertebral space until a stop on the device abuts against an outer surface of at least one of the vertebrae and prevents further insertion of the device.

32. The method of claim 27 further comprising aligning a vertebral plate with the distraction device and attaching the plate to at least one of the vertebrae after the engaging and varying steps.

33. The method of claim 27 wherein the varying step is used to adjust one of a lordotic, kyphotic or scoliotic angle of a portion of a spine.

34. The method of claim 27 wherein the varying step is used to simultaneously adjust more than one of a lordotic, kyphotic or scoliotic angle of a portion of a spine.
